(19) United States
(12) Patent Application Publication
(10) Pub. No.: US 2020/0078064 A1
(43) Pub. Date: Mar. 12, 2020

(54) BONE SEGMENT ATTACHMENT SYSTEM AND METHOD

(71) Applicant: InMotus Medical LLC, Carmel, IN (US)

(72) Inventors: Adam D. Perler, St. Peterburg, FL (US); James A. Zoellner, Avon, IN (US); James Q. Spitler, Ocoee, FL (US)

(73) Assignee: InMotus Medical LLC, Carmel, IN (US)

(21) Appl. No.: 16/565,482

(22) Filed: Sep. 9, 2019

Related U.S. Application Data
(60) Provisional application No. 62/729,245, filed on Sep. 10, 2018.

(51) Int. Cl. A61B 17/86; A61F 2/42

(52) U.S. Cl. CPC A61B 17/8625; A61B 2017/564; A61F 2/4225; A61F 2/4241

(57) ABSTRACT

A system for attaching a middle phalanx and a proximal phalanx together may include a middle component and a proximal component. The middle component may have a middle component bone fastener implantable into a first bore formed in the middle phalanx, and a middle component attachment end with a first engagement feature with a first shape. The proximal component may have a proximal bone fastener implantable into a second bore formed in the proximal phalanx, and a proximal component attachment end with a second engagement feature with a second shape. The first engagement feature may be movable into engagement with the second engagement feature along an engagement direction that is generally perpendicular to the middle phalanx and/or the proximal phalanx. The second shape may be complementary to the first shape such that the first engagement feature interlocks with the second engagement feature.

### RELATED APPLICATIONS

This application claims the benefit of U.S. Provisional Patent Application Ser. No. 62/729,245, filed on Sep. 10, 2018 and entitled BONE SEGMENT ATTACHMENT SYSTEM AND METHOD. The foregoing is incorporated by reference as though set forth herein in its entirety.

### TECHNICAL FIELD

The present disclosure relates to surgical systems, and methods. More specifically, the present disclosure relates to improved surgical implants for securing a first bone segment, such as a proximal phalanx, to a second bone segment, such as a middle phalanx.

### BACKGROUND

There are many instances in which two bone segments of the body are to be secured together. For example, fragments of a broken bone may be secured through the use of surgical implants. Alternatively, joints between bones may be effectively immobilized to alleviate pain or correct deformities by securing the bone segments on either side of the joint together.

Current technologies for attaching bone segments together may be insufficient to meet the needs of patients. Some known systems include male and female components that can only be secured together after excessive distraction of the joint has been carried out, to align the male and female components together. Such excessive distraction may harm the soft tissues surrounding the joint, and may extend the patient's recovery time. Further, such distraction may make it more difficult to revise the joint arthrodesis procedure in the future.

Furthermore, some known bone segment attachment systems may not provide sufficiently secure fixation to prevent the bone segments from moving relative to each other, after implantation of the bone segment attachment system. Such motion may inhibit proper bone fusion and/or healing.

### SUMMARY

The various systems and methods of the present disclosure have been developed in response to the present state of the art, and in particular, in response to the problems and needs in the art that have not yet been fully solved by currently available surgical instruments, systems, and methods.

According to one embodiment, a system for attaching a middle phalanx and a proximal phalanx together may include a middle component and a proximal component. The middle component may have a middle component bone fastener implantable into a first bore formed in the middle phalanx, and a middle component attachment end with a first engagement feature with a first shape. The proximal component may have a proximal bone fastener implantable into a second bore formed in the proximal phalanx, and a proximal component attachment end with a second engagement feature with a second shape. The first engagement feature may be movable into engagement with the second engagement feature along an engagement direction that is generally perpendicular to the middle phalanx and/or the proximal phalanx. The second shape may be complementary to the first shape such that the first engagement feature interlocks with the second engagement feature in a manner that restricts withdrawal of the first engagement feature from the second engagement feature along any direction nonparallel to the engagement direction. One of the middle component attachment end and the proximal component attachment end may further have a locking feature with an unlocked configuration in which the first engagement feature is movable out of engagement with the second engagement feature, and a locked configuration in which the locking feature blocks motion of the first engagement feature out of engagement with the second engagement feature.

The engagement direction may be from a dorsal aspect of the middle phalanx and the proximal phalanx toward a plantar aspect of the middle phalanx and the proximal phalanx. The engagement direction may be perpendicular to one of the middle bone fastener and the proximal bone fastener. The engagement direction may be disposed at an angle relative to the other of the middle bone fastener and the proximal bone fastener. The angle may be within the range of 60° to 85°.

One of the first engagement feature and the second engagement feature may be a slot. The other of the first engagement feature and the second engagement feature may be a plateau shaped to be insertable into the slot along the engagement direction. One of the middle component attachment end and the proximal component attachment end may further have a resilient lip with a deflected state in which the plateau is withdrawable from the slot, and relaxed state in which the resilient lip blocks withdrawal of the plateau from the slot.

According to one embodiment, a system for attaching a middle phalanx and a proximal phalanx together may include a middle component and a proximal component. The middle component may have a middle bone fastener implantable in a first bore in the middle phalanx, and a middle component attachment end with a first engagement feature. The proximal component may have a proximal bone fastener implantable in a second bore in the proximal phalanx, and a proximal component attachment end with a second engagement feature. The first engagement feature may be movable into engagement with the second engagement feature along an engagement direction that is nonparallel to the first bore and the second bore.

The engagement direction may be generally perpendicular to the middle phalanx and/or the proximal phalanx.

The engagement direction may be from a dorsal aspect of the middle phalanx and the proximal phalanx toward a plantar aspect of the middle phalanx and the proximal phalanx.

The engagement direction may be perpendicular to one of the middle bone fastener and the proximal bone fastener. The engagement direction may be disposed at an angle relative to the other of the middle bone fastener and the proximal bone fastener. The angle may be within the range of 60° to 85°.

The first engagement feature may be movable into engagement with the second engagement feature with the middle bone fastener implanted in the first bore, and with the proximal bone fastener implanted in the second bore.

One of the middle component attachment end and the proximal component attachment end may further have a locking feature with an unlocked configuration in which the first engagement feature is movable out of engagement with the second engagement feature, and a locked configuration in which the locking feature blocks motion of the first engagement feature out of engagement with the second engagement feature.

The first engagement feature may have a first shape. The second engagement feature may have a second shape. The second shape may be complementary to the first shape such that the first engagement feature interlocks with the second engagement feature in a manner that restricts withdrawal of the first engagement feature from the second engagement feature along any direction nonparallel to the engagement direction.

One of the first engagement feature and the second engagement feature may be a slot. The other of the first engagement feature and the second engagement feature may be a plateau shaped to be insertable into the slot along the engagement direction.

One of the proximal component attachment end and the middle component attachment end may further have a resilient lip with a deflected state in which the plateau is withdrawable from the slot, and relaxed state in which the resilient lip blocks withdrawal of the plateau from the slot.

According to one embodiment, a method for attaching a middle phalanx and a proximal phalanx together may utilize a system with a middle component and a proximal component. The middle component may have a middle component bone fastener and a middle component attachment end. The proximal component may have a proximal component bone fastener and a proximal component attachment end. The method may include implanting the middle component bone fastener in a first bore in the middle phalanx, implanting the proximal component bone fastener in a second bore in the proximal phalanx, and, with the middle component bone fastener implanted in the first bore and the proximal component bone fastener implanted in the second bore, moving a first engagement feature of the middle component attachment end into engagement with a second engagement feature of the proximal component attachment end, along an engagement direction that is nonparallel to the first bore and the second bore.

Moving the first engagement feature into engagement with the second engagement feature along the engagement direction may include moving at least one of the first engagement feature and the second engagement feature generally perpendicular to the middle phalanx and/or the proximal phalanx.

Moving the first engagement feature into engagement with the second engagement feature along the engagement direction may include moving the first engagement feature, relative to the second engagement feature, from a dorsal aspect of the middle phalanx and the proximal phalanx toward a plantar aspect of the middle phalanx and the proximal phalanx.

The engagement direction may be perpendicular to one of the middle component bone fastener and the proximal component bone fastener. The engagement direction may be disposed at an angle relative to the other of the middle component bone fastener and the proximal component bone fastener. The angle may be within the range of 60° to 85°.

One of the middle component attachment end and the proximal component attachment end may further have a locking feature with an unlocked configuration and a locked configuration. Moving the first engagement feature into engagement with the second engagement feature may include moving the first engagement feature into engagement with the second engagement feature with the locking feature in the unlocked configuration. The method may further include, after moving the first engagement feature into engagement with the second engagement feature, moving the locking feature to the locked configuration to block motion of the first engagement feature out of engagement with the second engagement feature.

The first engagement feature may have a first shape. The second engagement feature may have a second shape. The second shape may be complementary to the first shape such that the first engagement feature interlocks with the second engagement feature in a manner that restricts withdrawal of the first engagement feature from the second engagement feature along any direction nonparallel to the engagement direction.

One of the first engagement feature and the second engagement feature may be a slot. The other of the first engagement feature and the second engagement feature may be a plateau. Moving the first engagement feature into engagement with the second engagement feature may include inserting the plateau into the slot along the engagement direction.

One of the middle component attachment end and the proximal component attachment end may further have a resilient lip with a deflected state and a relaxed state. Inserting the plateau into the slot may include inserting the plateau into the slot with the resilient lip in the deflected state. The method may further include, after moving the plateau into the slot, moving the resilient lip to the relaxed state to block motion of the plateau from the slot.

Additional details will be set forth in the detailed description and drawings.

### BRIEF DESCRIPTION OF THE DRAWINGS

Exemplary embodiments of the disclosure will become more fully apparent from the following description and appended claims, taken in conjunction with the accompanying Figures. Understanding that these Figures depict only exemplary embodiments and are, therefore, not to be considered limiting of the scope of the appended claims, the exemplary embodiments of the present disclosure will be described with additional specificity and detail through use of the accompanying Figures:

FIG. 1 is a perspective view of one exemplary embodiment of a bone segment attachment system.

FIG. 2 is a perspective view of the bone segment attachment system in greater detail.

FIG. 3 is a perspective view of the middle component of FIG. 2, in isolation.

FIG. 4 is a perspective view of the middle component attachment end of the middle component of FIG. 2 in isolation, without the middle component bone fastener.

FIG. 5 is a perspective view of the middle component bone fastener of the middle component attachment end of FIG. 2, in isolation.

FIG. 6 is a perspective view of the proximal component of FIG. 2, in isolation.

FIG. 7 is a perspective view of the proximal component attachment end of the proximal component in isolation, without the proximal component bone fastener.

FIG. 8 is an exploded, perspective view depicting the middle component attachment end and the proximal component attachment end, prior to attachment of the middle component attachment end to the proximal component attachment end.

FIG. 9 is a perspective view depicting the middle component attachment end and the proximal component attachment end, after to attachment of the middle component attachment end to the proximal component attachment end.

FIG. 10 is a flow diagram depicting a method of implanting the bone segment attachment system of FIGS. 1 through 9, according to one exemplary embodiment.

FIG. 11 is a flow diagram depicting a method of decoupling the first bone segment from the second bone segment after implantation of the bone segment attachment system, as in the method of FIG. 10, according to one exemplary embodiment.

### DETAILED DESCRIPTION

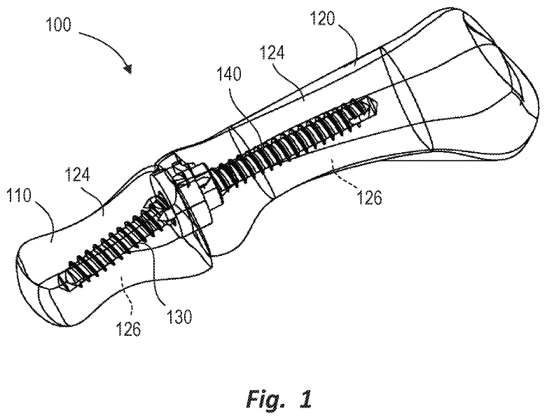

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method, as represented in FIGS. 1 through 8, is not intended to limit the scope of the disclosure, but is merely representative exemplary of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The present disclosure discloses bone segment attachment systems and methods. Such bone segment attachment systems and methods may be designed to attach one bone segment to another bone segment. A bone segment at least includes either a full or partial portion of a bone. In one example, bone segments include two phalanges, while in other u examples, the bone segments are other bones in body that are to be fused together, such as the bones of a joint that is to be immobilized. In yet other examples, the bone segments may include two segments of a bone that has been broken. Of course, one skilled in the art may recognize other situations where two bone segments may be attached together; this disclosure contemplates all such situations.

Current technologies for attaching bone segments together may be insufficient to meet the needs of patients. Some known systems include male and middle components that can only be secured together in a limited number of relative axial positions. A first relative axial position may not sufficiently place the two bone segments in contact with each other to allow them to fuse while an adjacent axial position may not be attainable due to interference of the bone segments with each other. Thus, in some situations, the resolution with which the male and middle components are attachable to each other may be insufficient to provide the desired bone in-growth between the two bone segments.

Furthermore, some known bone segment attachment systems may not provide sufficiently secure fixation to prevent the bone segments from moving relative to each other, after implantation of the bone segment attachment system. Such motion may inhibit proper bone fusion and/or healing.

FIG. 1 is a perspective view depicting one exemplary embodiment of a bone segment attachment system 100. In this embodiment, the bone segment attachment system 100 has been installed to affix a first bone segment 110 and a second bone segment 120 together. In one example, the bone segment attachment system 100 is installed to ensure contact between a surface of the first bone segment 110 and a surface of the second bone segment 120. Such contact allows the first bone segment 110 to fuse to the second bone segment 120 over a healing period of time.

The bone segment attachment system 100 may be used for a wide variety of joints or fractured bones in the body. As shown in FIG. 1, the bone segment attachment system 100 is used for an extremity, namely, the joint between the proximal and middle phalanges. Thus, the first bone segment 110 may be the middle phalanx and the second bone segment 120 may be the proximal phalanx of a human foot. In such a configuration, the bone segment attachment system 100 may be used to treat a condition such as hammertoe, for example, by fusing the joint between the proximal and middle phalanges such that the middle phalanx is secured in an anatomically natural orientation relative to the middle phalanx. The first bone segment 110 and the second bone segment 120 may have a dorsal aspect 124 representing the top of the patient's foot, and a plantar aspect 126 representing the bottom of the patient's foot.

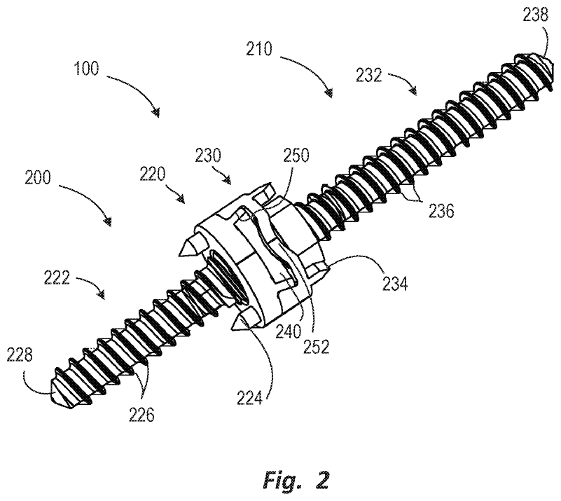

The bone segment attachment system 100 may be anchored in a first bore 130 of the first bone segment 110, and in a second bore 140 of the second bone segment 120. The bone segment attachment system 100 will be shown and described in greater detail in connection with FIGS. 2 through 9, as follows. These drawings and the accompanying description relate to one exemplary embodiment in which the first bone segment 110 is a distal phalanx and the second bone segment 120 is a middle phalanx. Those of skill in the art will recognize that the features disclosed herein may, in alternative embodiments, be used for other bone segments and/or joints.

Referring to FIG. 2, a perspective view depicts the bone segment attachment system 100 in greater detail. In one exemplary embodiment, the bone segment attachment system 100 has a middle component 200 securable to the first bone segment 110 and a proximal component 210 securable to the second bone segment 120. Notably, the descriptors “middle component” and “proximal component” are associated with the phalanges, consistent with an exemplary embodiment in which the first bone segment 110 is a distal phalanx and the second bone segment 120 is a middle phalanx. In alternative embodiments, the systems and methods of the present disclosure may be used in connection with other joints of the body.

Returning to the components of FIG. 2, the middle component 200 may have a middle component attachment end 220 that attaches the middle component 200 to the proximal component 210, and a middle component bone anchoring end 222 that attaches the middle component 200 to the first bone segment 110. Similarly, the proximal component 210 may have a proximal component attachment end 230 that attaches the proximal component 210 to the middle component 200, and a proximal component bone anchoring end 232 that attaches the proximal component 210 to the second bone segment 120.

The middle component bone anchoring end 222 and the proximal component bone anchoring end 232 may each have one or more bone engagement features configured to engage the corresponding bone segment. In the exemplary embodiment of FIG. 2, these bone engagement features are middle component helical threads 226 and proximal component helical threads 236 whereby a surgeon implanting the bone segment attachment system 100 may screw the middle component 200 into the first bore 130 of the first bone segment 110 and may screw the proximal component 210 into the second bore 140 of the second bone segment 120. In alternative embodiments, the bone engagement features are ridges, barbs, deployable extensions, expandable elements, and/or any other bone engagement features known in the art, for securing an implant to bone in a manner that resists withdrawal of the implant from the bone. As shown, the middle component helical threads 226 may be on a middle component bone fastener 228 that is a separate piece from the middle component attachment end 220, and the proximal component helical threads 236 may be on a proximal component bone fastener 238 that is a separate piece from the proximal component attachment end 230.

The middle component attachment end 220 may have projections 224 that project into the surface of the first bone segment 110 that faces toward the second bone segment 120. As embodied in FIG. 2, the projections 224 may be generally cylindrical in shape, with conical tips that penetrate the cortical bone of the first bone segment 110 as the middle component 200 is inserted into the first bore 130. Similarly, the proximal component attachment end 230 may have projections 234 that project into the surface of the second bone segment 120 that faces toward the first bone segment 110. As embodied in FIG. 2, the projections 234 may be rectangular prisms in shape, with triangular tips that penetrate the cortical bone of the second bone segment 120 as the proximal component 210 is inserted into the second bore 140. Notably, the shapes of the projections 224 and the projections 234 are merely exemplary; a wide variety of shapes may be used for projections to penetrate bone.

In one exemplary embodiment, a surgeon may first resect the adjoining ends of the first bone segment 110 and the second bone segment 120 to remove a natural joint between the first bone segment 110 and the second bone segment 120 and expose the adjoining bony surfaces of the first bone segment 110 and the second bone segment 120. The surgeon may then form the first bore 130 in the first bone segment 110 and form the second bore 140 in the second bone segment 120.

Once the first bone segment 110 and the second bone segment 120 have been prepared, the surgeon may then insert the middle component bone fastener 228 partially into the first bore 130 of the first bone segment 110 and rotate the middle component bone fastener 228 to cause the middle component helical threads 226 to engage the first bore 130. The surgeon may tighten the middle component bone fastener 228 to draw the middle component attachment end 220 against the exposed surface of the first bone segment 110 that faces the second bone segment 120, such that the projections 224 are drawn toward, and embedded in, the exposed surface of the first bone segment 110.

Similarly, the surgeon may insert the proximal component bone fastener 238 partially into the second bore 140 of the second bone segment 120 and rotate the proximal component bone fastener 238 to cause the proximal component helical threads 236 to engage the second bore 140. The surgeon may tighten the proximal component bone fastener 238 to draw the proximal component attachment end 230 against the exposed surface of the second bone segment 120 that faces the first bone segment 110, such that the middle component helical threads 226 are drawn toward, and embedded in, the exposed surface of the second bone segment 120.

With the middle component 200 and the proximal component 210 secured to the first bone segment 110 and the second bone segment 120, as described above, the surgeon may secure the middle component 200 to the proximal component 210. Specifically, the middle component 200 and the proximal component 210 may each have engagement features that engage each other to secure the middle component attachment end 220 to the proximal component attachment end 230.

In this application, an “engagement feature” is any type of feature that is made to facilitate the temporary or permanent coupling, together, of two separate parts. Engagement features may include any known mechanical fastening devices, including but not limited to clips, clasps, tongue-in-groove systems, threaded fasteners, combinations thereof, and the like. Moving one engagement feature into engagement with another encompasses any relative motion between the two engagement features, including motion in which (1) the first engagement feature is stationary while the other engagement feature moves, (2) the other engagement feature is stationary while the first engagement feature moves, and (3) both engagement features move.

A “locking feature” is any type of feature that is made to selectively lock or unlock a coupling between two parts. Thus, a locking feature may have a locked configuration, in which the locking feature keeps the two parts from being decoupled from each other, and an unlocked configuration, in which the locking feature allows the two parts to be decoupled from each other. Optionally, in the locked configuration, the locking feature may prevent the parts that have not yet been coupled together from being coupled together. Similarly, in the unlocked configuration, a locking feature may optionally allow parts to be coupled together.

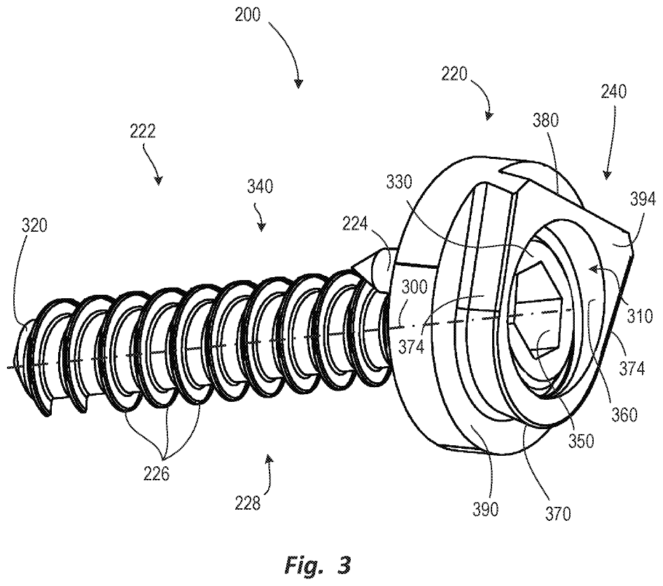

As shown in FIG. 2, the engagement features of the bone segment attachment system 100 may include a plateau 240 and a slot 250, which may be positioned adjacent to a locking feature in the form of a resilient lip 252. The plateau 240 may be on the middle component 200, and the slot 250 and the resilient lip 252 may be on the proximal component 210. The configuration and operation of these engagement features will be shown and described in greater detail in connection with FIGS. 3 through 9. FIG. 2 depicts the configuration in which the middle component 200 and the proximal component 210 are secured together via interlocking of the plateau 240 with the slot 250.

FIG. 3 is a perspective view of the middle component 200 of FIG. 2, in isolation. The middle component bone fastener 228 and the middle component attachment end 220 are shown in greater detail. The middle component 200 may have an axis 300.

As shown, the middle component bone fastener 228 may have a proximal end 310 and a distal end 320. The proximal end 310 may be configured to be engaged with tool such as a driver to facilitate rotation of the middle component bone fastener 228 within the first bore 130 of the first bone segment 110. As shown in FIG. 3, the proximal end 310 may have a head 330, which may be enlarged relative to a shank 340 that extends distally of the head 330 to the distal end 320. The head 330 may have a drive feature that engages the driver; as embodied in FIG. 3, the drive feature may be a hexagonal socket 350 that receives a hexagonal protrusion of the driver.

The middle component attachment end 220 may have a hole 360 that extends through the plateau 240, through which the head 330 of the middle component bone fastener 228 is exposed. Thus, the middle component attachment end 220 may be held against the first bone segment 110, and the middle component bone fastener 228 may be driven into the first bore 130 without the need to remove or subsequently re-attach the middle component attachment end 220.

The plateau 240 may have a leading end 370, sides 374, and a trailing end 380. The leading end 370 may be inserted first into the slot 250 of the proximal component attachment end 230, followed by the sides 374. The trailing end 380 may enter the slot 250 afterward, and may be retained by the resilient lip 252 of the proximal component attachment end 230. The middle component attachment end 220 may also have a shoulder 390 with a generally planar shape that extends outward from the distal edge of the plateau 240, and a proximal surface 394.

The leading end 370 and the sides 374 of the plateau 240 may have a dovetail shape such that the proximal edges of the leading end 370 and the sides 374 are relatively further from the axis 300. Consequently, the leading end 370 and the sides 374 may be non-parallel to the axis 300, and may face partially toward the shoulder 390. This dovetail shape may facilitate interlocking of the plateau 240 with the slot 250 of the proximal component attachment end 230, as will be shown and described subsequently. The trailing end 380 may optionally lack such a dovetail shape, and may rather be generally parallel to the axis 300.

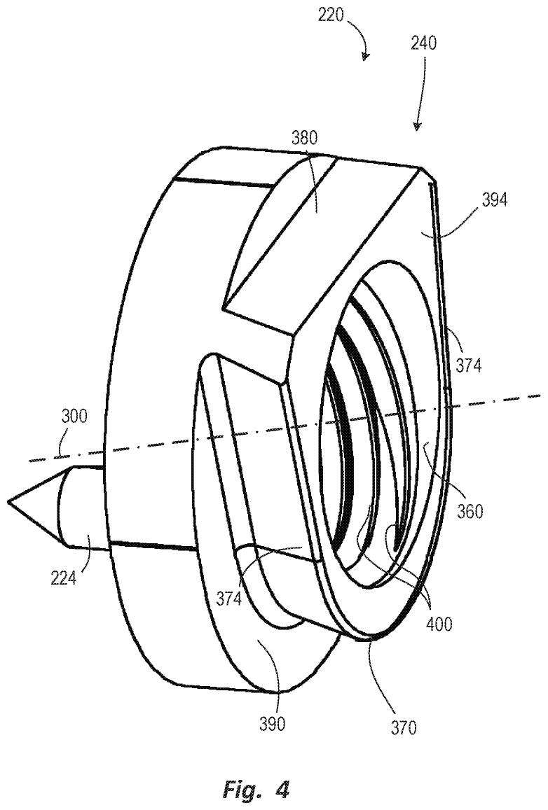

FIG. 4 is a perspective view of the middle component attachment end 220 of the middle component 200 in isolation, without the middle component bone fastener 228. As shown, the hole 360 of the middle component attachment end 220 has interior threads 400, which may mate with exterior threads on the head 330 of the middle component bone fastener 228, as will be shown and described subsequently. In alternative embodiments, the interior threads 400 may be replaced with a shoulder that is formed as part of a countersink or counterbore within the hole 360. Such features may keep the head 330 in place within the hole 360 without the need for threading. In such an embodiment, the middle component bone fastener 228 may not have corresponding threads on the head 330, but may rather have a shoulder on the head 330, adjoining the shank 340, that resides in the countersink or counterbore.

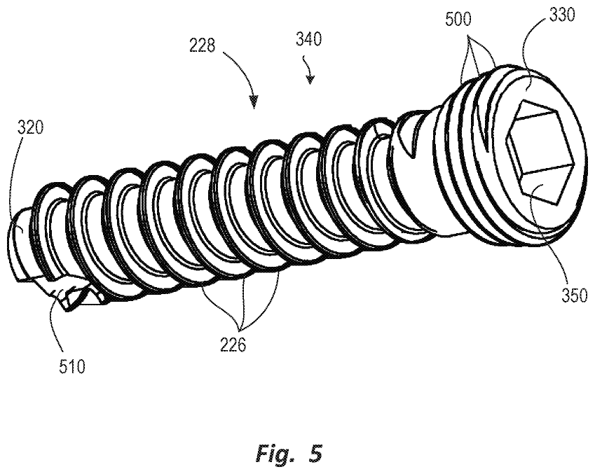

FIG. 5 is a perspective view of the middle component bone fastener 228 of the middle component bone anchoring end 222, in isolation. As shown, in addition to the features mentioned previously, the head 330 of the middle component bone fastener 228 may have exterior threads 500 that are sized and arranged to mate with the interior threads 400 of the hole 360 of the middle component attachment end 220. In operation, the middle component helical threads 226 of the shank 340 may engage the first bore 130 of the first bone segment 110 while the exterior threads 500 remain proximal to the interior threads 400 of the middle component attachment end 220. As the shank 340 is driven distally into the first bore 130, the middle component helical threads 226 fully engage with the first bore 130 and the exterior threads 500 engage the interior threads 400. As the shank 340 fully seats in the first bore 130, the exterior threads 500 may provide secure engagement with the interior threads 400 so that the middle component attachment end 220 is substantially fixed, relative to the head 330 of the middle component bone fastener 228.

Notably, the pitch of the interior threads 400 and the exterior threads 500 may be smaller than the pitch of the middle component helical threads 226. Thus, as the middle component bone fastener 228 is driven into the first bore 130, the middle component attachment end 220 may be driven toward the exposed surface of the first bone segment 110, on which is has been placed, with a lower velocity than the middle component bone fastener 228. Thus, the projection 224 may be driven relatively slowly, and with higher mechanical advantage, into the cortical bone of the first bone segment 110. This may facilitate penetration of the bone by the projection 224 and make it easier for the surgeon to fully seat the middle component attachment end 220 against the exposed surface of the first bone segment 110.

Further, the middle component bone fastener 228 may have a cutting feature in the form of a relief 510 that may facilitate insertion of the middle component bone fastener 228 into the first bore 130 of the first bone segment 110. For example, during insertion of the middle component bone fastener 228 into the first bore 130 of the first bone segment 110, the relief 510 may help dislodged bone matter and/or fluids to move from in front of the distal end 320 of the advancing middle component bone fastener 228 to the spaces between the middle component helical threads 226 of the middle component bone fastener 228, and thence, out of the first bore 130. The relief 510 is shown placed proximate the distal end 320; however, this location is merely exemplary, as one skilled in the art may place one or more reliefs in different locations on a bone screw to facilitate insertion of the screw and/or removal of tissue from the hole in which the screw is inserted.

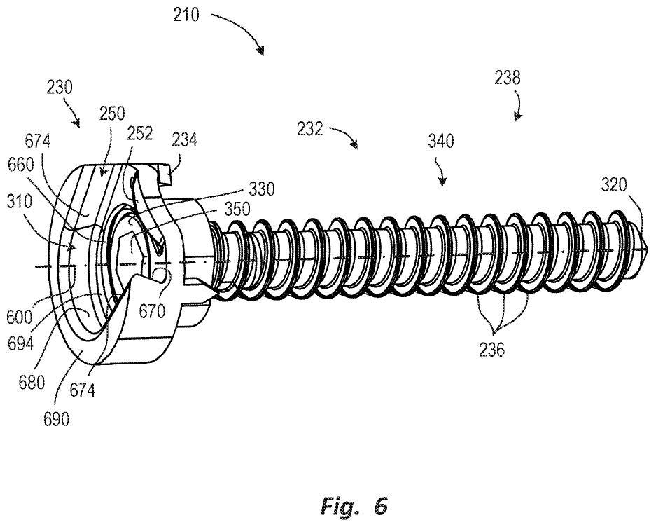

FIG. 6 is a perspective view of the proximal component 210 of FIG. 2, in isolation. The proximal component bone fastener 238 and the proximal component attachment end 230 are shown in greater detail. The proximal component 210 may have an axis 600.

As shown, the proximal component bone fastener 238 may be identical or similar to the middle component bone fastener 228. Thus, the proximal component bone fastener 238 may have a proximal end 310 and a distal end 320. The proximal end 310 may be configured to be engaged with tool such as a driver to facilitate rotation of the proximal component bone fastener 238 within the second bore 140 of the second bone segment 120. As shown in FIG. 6, the proximal end 310 may have a head 330, which may be enlarged relative to a shank 340 that extends distally of the head 330 to the distal end 320. The head 330 may have a drive feature that engages the driver; as embodied in FIG. 6, the drive feature may be a hexagonal socket 350 that receives a hexagonal protrusion of the driver.

The proximal component attachment end 230 may have a hole 660 that extends through into the slot 250, through which the head 330 of the proximal component bone fastener 238 is exposed. Thus, the proximal component attachment end 230 may be held against the second bone segment 120, and the proximal component bone fastener 238 may be driven into the second bore 140 without the need to remove or subsequently re-attach the proximal component attachment end 230.

The slot 250 may have a leading end 670, sides 674, and a trailing end 680. The proximal component attachment end 230 may further have a distal surface 690 that generally encircles the distal edge of the slot 250, and a shoulder 694 that extends between the hole 660 and the proximal edge of the slot 250. The trailing end 680 and the sides 674 of the slot 250 may have a dovetail shape such that the distal edges of the trailing end 680 and the sides 674 are relatively closer the axis 600. Consequently, the trailing end 680 and the sides 674 may be non-parallel to the axis 600, and may face partially toward the shoulder 694. This dovetail shape may facilitate interlocking of the plateau 240 with the slot 250 of the proximal component attachment end 230.

The leading end 670 may receive the plateau 240 first, and then the plateau 240 may be moved along the sides 674 and to the trailing end 680. Notably, the resilient lip 252 may, in its un-deflected state, block entry of the end 370 of the plateau 240 into the end 670 of the slot 250. Prior to insertion of the plateau 240 into the slot 250, the proximal surface 394 of the plateau 240 may be pressed against the resilient lip 252 such that the proximal surface 394 presses the resilient lip 252 into a more planar configuration, permitting the plateau 240 to align with the slot 250. This force, urging the middle component attachment end 220 and the proximal component attachment end 230 together, may continue to be applied as the plateau 240 is inserted into the slot 250.

When the plateau 240 has been fully inserted into the slot 250, the leading end 370 of the plateau 240 may rest against the trailing end 680 of the slot 250, the sides 374 of the plateau 240 may rest against the sides 674 of the slot 250, and the trailing end 380 of the plateau 240 may reside within the leading end 670 of the slot 250. The proximal surface 394 of the plateau 240 may be generally coplanar with the shoulder 694 of the proximal component attachment end 230, and the shoulder 390 of the plateau 240 may abut the distal surface 690 of the proximal component attachment end 230. The resilient lip 252 may spring back into an undeflected, or non-planar, configuration once the trailing end 380 of the plateau 240 has moved beyond the edge of the resilient lip 252. Thus, the resilient lip 252 may abut the trailing end 380 to prevent withdrawal of the plateau 240 from the slot 250 in the absence of some force tending to bend the resilient lip 252 back into a deflected (more planar) configuration. The undeflected configuration may be referred to as a “locked” configuration, and the deflected (more planar) configuration may be referred to as an “unlocked” configuration.

The plateau 240, the slot 250, and the resilient lip 252 are merely examples of engagement features and locking features that may be used within the scope of the present disclosure. In alternative embodiments, various alternative engagement features and alternative locking features, including but not limited to clips, clamps, bayonet fittings, dovetails, threaded fasteners, deflecting fasteners, and the like may be used in place of or in addition to the plateau 240, the slot 250, and the resilient lip 252. Such engagement features and locking features may facilitate attachment of a middle component attachment end to a proximal component attachment end, and may facilitate selective locking of such attachment.

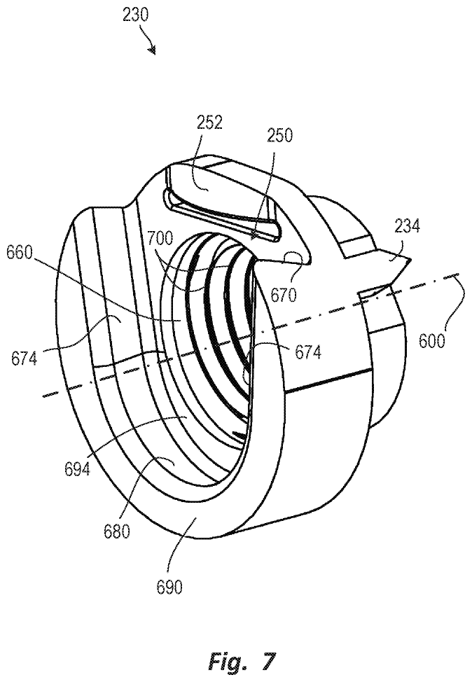

FIG. 7 is a perspective view of the proximal component attachment end 230 of the proximal component 210 in isolation, without the proximal component bone fastener 238. As shown, the hole 660 of the proximal component attachment end 230 has interior threads 700, which may mate with exterior threads 500 on the head 330 of the proximal component bone fastener 238, as shown and described in connection with the middle component bone fastener 228 of FIG. 5. In alternative embodiments, the interior threads 700 may be replaced with a shoulder that is formed as part of a countersink or counterbore within the hole 660. Such features may keep the head 330 in place within the hole 660 without the need for threading. In such an embodiment, the proximal component bone fastener 238 may not have corresponding threads on the head 330, but may rather have a shoulder on the head 330, adjoining the shank 340, that resides in the countersink or counterbore.

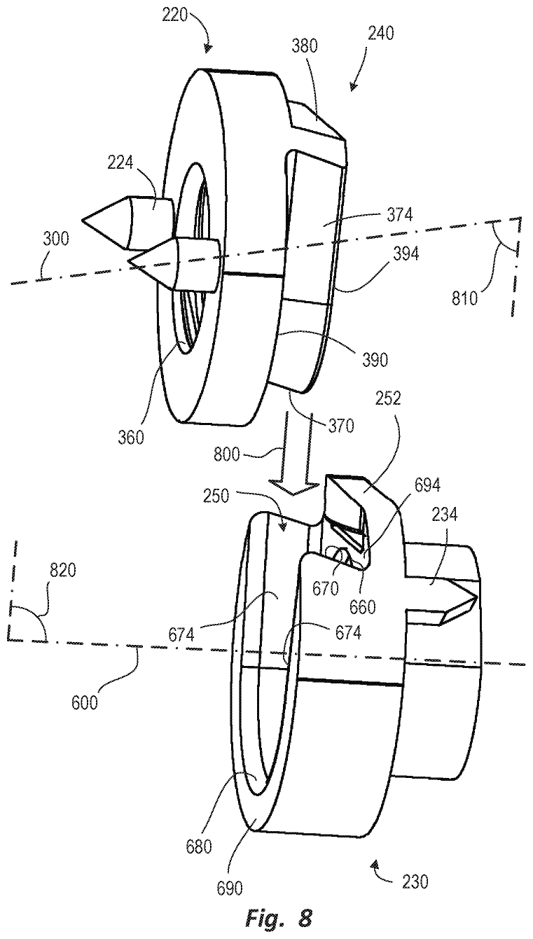

FIG. 8 is an exploded, perspective view depicting the middle component attachment end 220 and the proximal component attachment end 230, prior to attachment of the middle component attachment end 220 to the proximal component attachment end 230. As mentioned before, prior to securing the middle component attachment end 220 to the proximal component attachment end 230, the middle component 200 may be secured to the first bone segment 110, and the proximal component 210 may be secured to the second bone segment 120. The first bone segment 110, the second bone segment 120, the middle component bone fastener 228, and the proximal component bone fastener 238 are omitted from FIG. 8 for clarity.

An arrow 800 depicts the direction (the “engagement direction”) along which the plateau 240 may be moved into the slot 250. Notably, the arrow 800 is non-parallel to the first bone segment 110 and the second bone segment 120, and is also non-parallel to the first bore 130 and the second bore 140. Indeed, the arrow 800 may be generally perpendicular to the first bone segment 110 and the second bone segment 120, and may further be generally perpendicular to the first bore 130 and the second bore 140. This is distinct from other bone fusion systems in which the bones to be fused must be distracted significantly (i.e., drawn apart) and then inserted together along a direction perpendicular to a plane passing through the joint between the bones in order to provide the necessary mechanical interlock.

In some embodiments, it may be desirable to have the first bone segment 110 disposed at an angular offset relative to the second bone segment 120. Thus, the arrow 800 may be angled differently with respect to the axis 300 of the middle component 200 and the axis 600 of the proximal component 210. For example, the arrow 800 may be disposed at a first angle 810 relative to the axis 300 of the middle component 200, and at a second angle 820 relative to the axis 600 of the proximal component 210. The first angle 810 may be less than 90°, while the second angle 820 may be approximately 90°. Thus, the axis 300 may be positioned at an angular offset relative to the axis 600, to position the first bone segment 110 at a natural angle relative to the second bone segment 120. The first angle 810 may fall within the range of 45° to 87.5°. Further, the first angle 810 may fall within the range of 60° to 85°. Yet further, the first angle 810 may fall within the range of 70° to 80°. Thus, the first bone segment 110 may be offset from the second bone segment 120 by an angle of 10° to 20°.

In the bone segment attachment system 100 of FIG. 1, the angles of the middle component bone fastener 228 and the proximal component bone fastener 238 may be fixed relative to the middle component 200 and the proximal component 210, respectively. However, in alternative embodiments, the middle component bone fastener 228 may be coupled to the middle component 200 at an adjustable angle and/or the proximal component bone fastener 238 may be coupled to the proximal component 210 at an adjustable angle. For example, in place of the interior threads 400 of the middle component attachment end 220 and the interior threads 700 of the proximal component attachment end 230, polyaxial interfaces (not shown) may be used to provide polyaxial adjustability of the angles at which the middle component bone fastener 228 and the proximal component bone fastener 238 are coupled to the middle component 200 and the proximal component 210, respectively. Exemplary polyaxial interfaces include a ball-and socket system in which the head of each fastener has a semispherical shape that is received within a correspondingly-shaped socket of each component. Thus, a bone segment attachment system may accommodate different bone morphologies and/or desired bone attachment angles.

Returning to the bone segment attachment system 100 of FIG. 1, further shown in connection with FIG. 8, the plateau 240 and the slot 250 may advantageously come together in a manner that does not require the middle component 200 and the proximal component 210 to be drawn far apart from each other. Thus, only limited or no distraction of the joint space between the first bone segment 110 and the second bone segment 120 is needed. This may help avoid damage to soft tissues surrounding the joint. Additionally, the plateau 240 and the slot 250 may provide secure engagement in which the middle component 200 and the proximal component 210 are substantially unable to rotate relative to each other. Thus, the joint may remain in a natural and/or anatomic orientation, as selected by the surgeon.

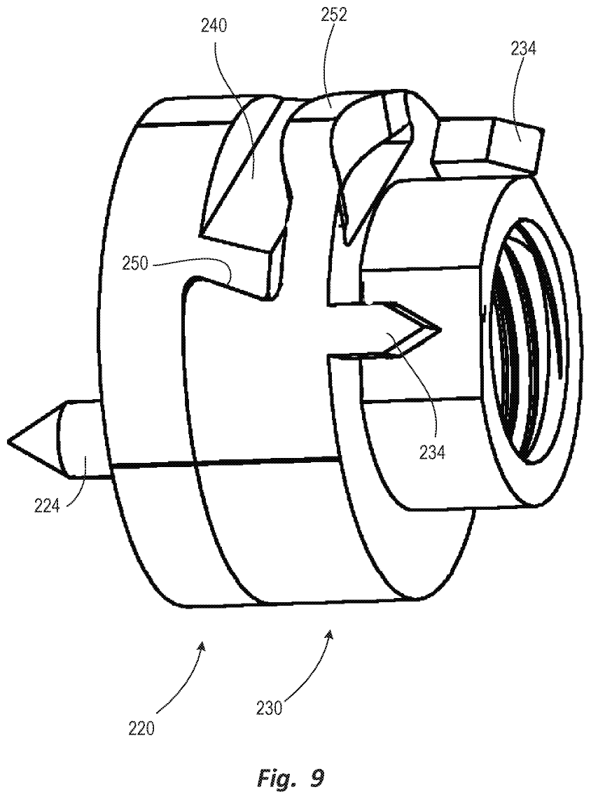

FIG. 9 is a perspective view depicting the middle component attachment end 220 and the proximal component attachment end 230, after to attachment of the middle component attachment end 220 to the proximal component attachment end 230. The middle component 200 and the proximal component 210 are now secured together to allow the first bone segment 110 and the second bone segment 120 to fuse together.

Optionally, the middle component 200 and the proximal component 210 may be generally compact in shape such that significant portions of the exposed surfaces of the first bone segment 110 and the second bone segment 120 are close enough to each other to permit fusion between the first bone segment 110 and the second bone segment 120. If desired, the first bore 130 and/or the second bore 140 may have a countersink, counterbore, or other feature, as depicted in FIG. 1, that permits the middle component attachment end 220 and/or the proximal component attachment end 230 to be recessed in the first bone segment 110 and/or the second bone segment 120. Such recessed positioning may enable the exposed surfaces of the first bone segment 110 and the second bone segment 120 to be positioned closer together to facilitate bone in-growth between them, and ultimately fusion of the joint between the first bone segment 110 and the second bone segment 120.

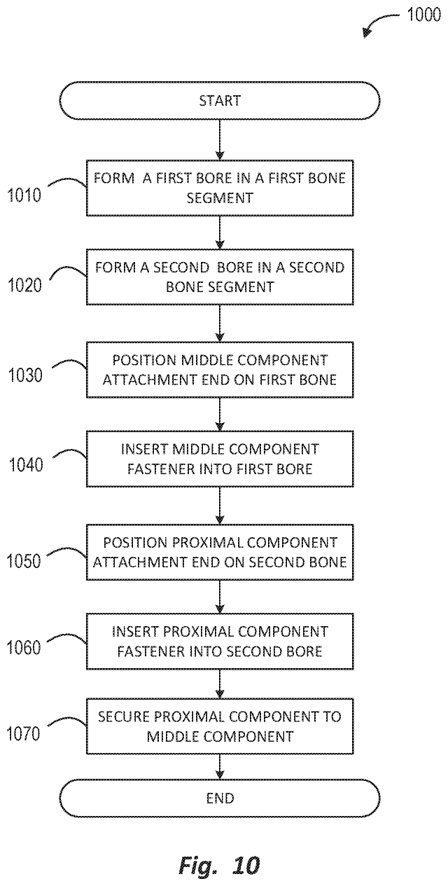

FIG. 10 is a flow diagram depicting a method 1000 of implanting the bone segment attachment system 100 of FIGS. 1 through 9, according to one exemplary embodiment. The method 1000 begins and at step 1010, in which the first bore 130 is formed in the first bone segment 110. In one example, a user may drill, ream, or otherwise form the first bore 130 through the use of an instrument. In alternative embodiments, the middle component bone anchoring end 222 may be designed to form the first bore 130 during insertion, such that the first bore 130 need not be formed prior to insertion of the middle component bone anchoring end 222 into the first bone segment 110. Rather, the process of inserting the middle component bone anchoring end 222 into the first bone segment 110 may form the first bore 130.

In a step 1020, the second bore 140 is formed in the second bone segment 120. As in the step 1010, the second bore 140 may be formed through the use of one or more tools known in the art, and/or through the use of bore-forming functionality in the proximal component bone anchoring end 232.

In a step 1030, the middle component attachment end 220 may be positioned on the exposed surface of the first bone segment 110, such that the hole 360 is aligned with the first bore 130 of the first bone segment 110. The middle component bone fastener 228 may be separate from the middle component attachment end 220 as the middle component attachment end 220 is positioned and held against the bone. The middle component bone fastener 228 may be positioned with the distal end 320 of the middle component bone fastener 228 at the opening of the first bore 130, or if the first bore 130 is to be formed via the middle component bone fastener 228, at the point at which the first bore 130 is to intersect the exposed surface of the first bone segment 110.

In a step 1040, the middle component bone fastener 228 is inserted into the first bore 130 of the first bone segment 110, or used to form the first bore 130 of the first bone segment 110, with the shank 340 of the middle component bone fastener 228 in place in the first bore 130. This may secure the middle component attachment end 220 to the first bone segment 110.

In a step 1050, the proximal component attachment end 230 may be positioned on the exposed surface of the second bone segment 120, such that the hole 660 is aligned with the second bore 140 of the second bone segment 120. The proximal component bone fastener 238 may be separate from the proximal component attachment end 230 as the proximal component attachment end 230 is positioned and held against the bone. The proximal component bone fastener 238 may be positioned with the distal end 320 of the proximal component bone fastener 238 at the opening of the second bore 140, or if the second bore 140 is to be formed via the proximal component bone fastener 238, at the point at which the second bore 140 is to intersect the exposed surface of the second bone segment 120. Care may be taken to align the orientation of the proximal component attachment end 230 with that of the middle component attachment end 220 as depicted in FIG. 8.

In a step 1060, the proximal component bone fastener 238 is inserted into the second bore 140 of the second bone segment 120, or used to form the second bore 140 of the second bone segment 120, with the shank 340 of the proximal component bone fastener 238 in place in the second bore 140. This may secure the proximal component attachment end 230 to the second bone segment 120.

In a step 1070, the proximal component attachment end 230 is secured to the middle component attachment end 220. This may be done as shown and described in connection with FIGS. 8 and 9, by inserting the plateau 240 of the middle component attachment end 220 into the slot 250 of the proximal component attachment end 230. The middle component 200 and the proximal component 210 may then be locked together, effectively locking the first bone segment 110 to the second bone segment 120 in a manner that permits fusion to occur between the first bone segment 110 and the second bone segment 120.

The surgical wound site may then be closed. According to an optional step, a brace, cast, or other device (not shown) may be attached to the patient's toe, of which the first bone segment 110 and the second bone segment 120 are part, to keep the first bone segment 110 in the desired position and orientation relative to the second bone segment 120 until bone in-growth can occur between the first bone segment 110 and the second bone segment 120.

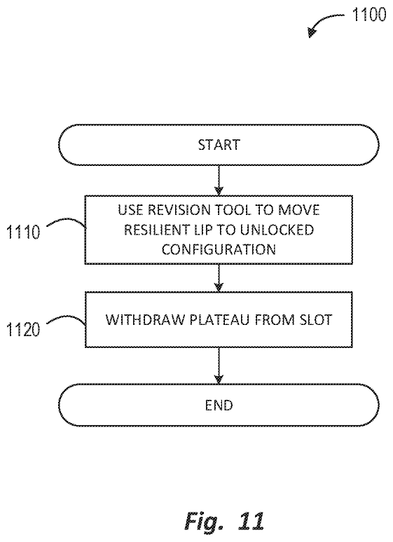

FIG. 11 is a flow diagram depicting a method 1100 of decoupling the first bone segment 110 from the second bone segment 120 after implantation of the bone segment attachment system 100, as in the method 1000 of FIG. 10, according to one exemplary embodiment. The method 1100 begins and at step 1110, in which a revision tool (not shown) or other tool is used to press proximally against the resilient lip 252, pushing it to the unlocked configuration.

In a step 1120, with the resilient lip 252 in the unlocked configuration, the plateau 240 of the middle component attachment end 220 may be withdrawn from the slot 250 of the proximal component attachment end 230. Once the plateau 240 is out of the slot 250, the joint and/or the bone segment attachment system 100 may be revised as needed.

Those of skill in the art will recognize that the various steps of the method 1000 and the method 1100 may be reordered, omitted, replaced with other steps, and/or supplemented with additional steps not specifically set forth herein. Although the method 1000 and the method 1100 are described in connection with the bone segment attachment system 100 of FIGS. 1 through 9, those of skill in the art will recognize that the method 1000 and the method 1100 could be carried out in connection with differently-configured bone attachment systems. Similarly, the bone segment attachment system 100 of FIGS. 1 through 9 may be used in connection with methods different from the method 1000 and the method 1100.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. A system for attaching a middle phalanx and a proximal phalanx together, the system comprising:
a middle component comprising:
a middle bone fastener implantable in a first bore in the middle phalanx; and
a middle component attachment end comprising a first engagement feature with a first shape; and
a proximal component comprising:
a proximal bone fastener implantable in a second bore in the proximal phalanx; and
a proximal component attachment end comprising a second engagement feature with a second shape;
wherein:
the first engagement feature is movable into engagement with the second engagement feature along an engagement direction that is generally perpendicular to the middle phalanx and/or the proximal phalanx;
the second shape is complementary to the first shape such that the first engagement feature interlocks with the second engagement feature in a manner that restricts withdrawal of the first engagement feature from the second engagement feature along any direction nonparallel to the engagement direction; and
one of the middle component attachment end and the proximal component attachment end further comprises a locking feature comprising:
an unlocked configuration in which the first engagement feature is movable out of engagement with the second engagement feature; and
a locked configuration in which the locking feature blocks motion of the first engagement feature out of engagement with the second engagement feature.

2. The system of claim 1, wherein:
the engagement direction is from a dorsal aspect of the middle phalanx and the proximal phalanx toward a plantar aspect of the middle phalanx and the proximal phalanx;
the engagement direction is perpendicular to one of the middle bone fastener and the proximal bone fastener;
the engagement direction is disposed at an angle relative to the other of the middle bone fastener and the proximal bone fastener; and
the angle is within the range of 60° to 85°.

3. The system of claim 1, wherein:
one of the first engagement feature and the second engagement feature comprises a slot;
the other of the first engagement feature and the second engagement feature comprises a plateau shaped to be insertable into the slot along the engagement direction; and
one of the middle component attachment end and the proximal component attachment end further comprises a resilient lip comprising a deflected state in which the plateau is withdrawable from the slot, and relaxed state in which the resilient lip blocks withdrawal of the plateau from the slot.

4. A system for attaching a middle phalanx and a proximal phalanx together, the system comprising:
a middle component comprising:
a middle bone fastener implantable in a first bore in the middle phalanx; and
a middle component attachment end comprising a first engagement feature; and
a proximal component comprising:
a proximal bone fastener implantable in a second bore in the proximal phalanx; and
a proximal component attachment end comprising a second engagement feature;
wherein the first engagement feature is movable into engagement with the second engagement feature along an engagement direction that is nonparallel to the first bore and the second bore.

5. The system of claim 4, wherein the engagement direction is generally perpendicular to the middle phalanx and/or the proximal phalanx.

6. The system of claim 5, wherein the engagement direction is from a dorsal aspect of the middle phalanx and the proximal phalanx toward a plantar aspect of the middle phalanx and the proximal phalanx.

7. The system of claim 4, wherein:
the engagement direction is perpendicular to one of the middle bone fastener and the proximal bone fastener;
the engagement direction is disposed at an angle relative to the other of the middle bone fastener and the proximal bone fastener; and
the angle is within the range of 60° to 85°.

8. The system of claim 4, wherein the first engagement feature is movable into engagement with the second engagement feature with the middle bone fastener implanted in the first bore, and with the proximal bone fastener implanted in the second bore.

9. The system of claim 4, wherein one of the middle component attachment end and the proximal component attachment end further comprises a locking feature comprising:
an unlocked configuration in which the first engagement feature is movable out of engagement with the second engagement feature; and
a locked configuration in which the locking feature blocks motion of the first engagement feature out of engagement with the second engagement feature.

10. The system of claim 4, wherein:
the first engagement feature comprises a first shape;
the second engagement feature comprises a second shape; and
the second shape is complementary to the first shape such that the first engagement feature interlocks with the second engagement feature in a manner that restricts withdrawal of the first engagement feature from the second engagement feature along any direction nonparallel to the engagement direction.

11. The system of claim 10, wherein:
one of the first engagement feature and the second engagement feature comprises a slot; and
the other of the first engagement feature and the second engagement feature comprises a plateau shaped to be insertable into the slot along the engagement direction.

12. The system of claim 11, wherein one of the proximal component attachment end and the middle component attachment end further comprises a resilient lip comprising a deflected state in which the plateau is withdrawable from the slot, and relaxed state in which the resilient lip blocks withdrawal of the plateau from the slot.

13. A method for attaching a middle phalanx and a proximal phalanx together with a system comprising:
a middle component comprising:
a middle component bone fastener; and
a middle component attachment end; and
a proximal component comprising:
a proximal component bone fastener; and
a proximal component attachment end;
wherein the method comprises:
implanting the middle component bone fastener in a first bore in the middle phalanx;
implanting the proximal component bone fastener in a second bore in the proximal phalanx; and
with the middle component bone fastener implanted in the first bore and the proximal component bone fastener implanted in the second bore, moving a first engagement feature of the middle component attachment end into engagement with a second engagement feature of the proximal component attachment end, along an engagement direction that is nonparallel to the first bore and the second bore.

14. The method of claim 13, wherein moving the first engagement feature into engagement with the second engagement feature along the engagement direction comprises moving at least one of the first engagement feature and the second engagement feature generally perpendicular to the middle phalanx and/or the proximal phalanx.

15. The method of claim 14, wherein moving the first engagement feature into engagement with the second engagement feature along the engagement direction comprises moving the first engagement feature, relative to the second engagement feature, from a dorsal aspect of the middle phalanx and the proximal phalanx toward a plantar aspect of the middle phalanx and the proximal phalanx.

16. The method of claim 13, wherein:
the engagement direction is perpendicular to one of the middle component bone fastener and the proximal component bone fastener;
the engagement direction is disposed at an angle relative to the other of the middle component bone fastener and the proximal component bone fastener; and
the angle is within the range of 60° to 85°.

17. The method of claim 13, wherein:
one of the middle component attachment end and the proximal component attachment end further comprises a locking feature comprising an unlocked configuration and a locked configuration;
moving the first engagement feature into engagement with the second engagement feature comprises moving the first engagement feature into engagement with the second engagement feature with the locking feature in the unlocked configuration; and
the method further comprises, after moving the first engagement feature into engagement with the second engagement feature, moving the locking feature to the locked configuration to block motion of the first engagement feature out of engagement with the second engagement feature.

18. The method of claim 13, wherein:
the first engagement feature comprises a first shape;
the second engagement feature comprises a second shape; and
the second shape is complementary to the first shape such that the first engagement feature interlocks with the second engagement feature in a manner that restricts withdrawal of the first engagement feature from the second engagement feature along any direction nonparallel to the engagement direction.

19. The method of claim 18, wherein:
one of the first engagement feature and the second engagement feature comprises a slot;
the other of the first engagement feature and the second engagement feature comprises a plateau; and
moving the first engagement feature into engagement with the second engagement feature comprises inserting the plateau into the slot along the engagement direction.

20. The method of claim 19, wherein:
one of the middle component attachment end and the proximal component attachment end further comprises a resilient lip comprising a deflected state and a relaxed state;
inserting the plateau into the slot comprises inserting the plateau into the slot with the resilient lip in the deflected state; and
the method further comprises, after moving the plateau into the slot, moving the resilient lip to the relaxed state to block motion of the plateau from the slot.
